(19) United States
(12) Patent Application Publication
(10) Pub. No.: US 2025/0145543 A1
(43) Pub. Date: May 8, 2025

(54) METHODS FOR THE PRODUCTION OF A SOLID UREA-THIOSULFATE FERTILIZER

(71) Applicant: TESSENDERLO GROUP NV, Brussels (BE)

(72) Inventors: Jeroen VAN CAUWENBERGH, Brussels (BE); Lien TELEN, Brussels (BE); Mark BROUWER, Maastricht (NL); Jan VANDENDRIESSCHE, Brussels (BE)

(73) Assignee: TESSENDERLO GROUP NV, Brussels (BE)

(21) Appl. No.: 18/839,108

(22) PCT Filed: Feb. 21, 2023
(86) PCT No.: PCT/EP2023/054332

§ 371 Date: Aug. 16, 2024

(30) Foreign Application Priority Data
Feb. 22, 2022 (EP) 22158044.2

(51) Int. Cl. C05C 9/00; C05C 3/00

(52) U.S. Cl. CPC C05C 9/005; C05C 3/005

(57) ABSTRACT

The present invention relates to methods for the production of a solid urea-thiosulfate fertilizer wherein the method comprises preparing a liquid urea-thiosulfate blend comprising 70-90 wt. % urea and 10-30 wt. % thiosulfate salt and processing this blend in liquid form. Various process steps such as evaporation and/or transport to the solidification apparatus can be performed with decreased energy usage and decreased thiosulfate decomposition since the melting point of the blend is significantly lower than the melting point of a regular urea melt.

### FIELD OF THE INVENTION

The present invention relates to methods for the production of a solid urea-thiosulfate fertilizer.

### BACKGROUND OF THE INVENTION

Urea (CO(NH₂)₂) is a known nitrogen fertilizer which has the highest nitrogen content of all solid nitrogenous fertilizers in common use. More than 90% of world industrial production of urea is destined for use as a fertilizer.

Industrial production of urea is well known and based on a high-temperature, high-pressure reaction of carbon dioxide and ammonia to form ammonium carbamate, and subsequent decomposition of the ammonium carbamate to form urea and water. This process is performed in dedicated urea synthesis plants, with optimized recycling of reagents and side-products and energy recuperation.

Generally, after urea synthesis, the aqueous urea solution obtained is passed through one or more recovery sections where it is concentrated and non-converted reactants are recovered and fed back to the synthesis reactor. The urea is typically concentrated to at least 95 wt. % urea before it is fed as a high-temperature melt to e.g. a prill tower or a granulator where it is solidified into small particles (e.g. prills or granules) under the influence of a gas stream, typically ambient air. The solidifying gas stream contains dust and ammonia released from the urea melt during the cooling and solidification process, and may be treated to reduce its dust and ammonia content before it is released into the atmosphere.

In the past years, efforts have been made to react ammonia waste streams obtained from various points in an urea synthesis process with sulphuric acid, converting it into ammonium sulphate, and recovering the ammonium sulphate for use as a fertilizer.

WO2006/004424 describes the production of urea-ammonium sulphate by in-situ reaction of sulphuric acid and ammonia in an aqueous urea solution.

WO2014/188371 describes the production of ammonium sulphate by reacting ammonia recovered from the gas stream of a solidification unit in a ureaplant with sulphuric acid.

WO2018/092057 describes the production of urea-ammonium sulphate wherein part of the ammonia is obtained from the recovery section of the urea synthesis plant and the resulting ammonium sulphate is combined with an urea melt.

Sulfur is part of the so-called secondary plant nutrients and like the primary nutrients (NPK), is essential for plant health and growth, although in lesser amounts than the primary nutrients. Sulfur is termed as the secondary nutrient only to refer to its quantity, not its importance in the healthy growth of the plants and crops. Sulfur is essential for nitrogen fixation in nodules on legumes, and it is necessary in the formation of chlorophyll. Plants use sulfur for producing proteins, amino acids, enzymes, and vitamins for a healthy growth. Sulfur generates resistance to disease. Most of the sulfur in soils is found in soil in organic matter. However, it is not available to plants in this form. In order to become available to plants, the sulfur must be first released from the organic matter and go through mineralization process. The mineralization process is a result of microbial activity. In this process sulfur is converted to the sulphate form (SO₄²⁻), which is readily available to plants. Oil crops, legumes, forages and some vegetable crops require sulfur in considerable amounts. In many crops, its amount in the plant is similar to phosphorus. Although it is considered a secondary nutrient, it is now becoming recognized as the ‘fourth macronutrient’, along with nitrogen, phosphorus and potassium. Sulfur deficiency symptoms show up as light green to yellowish color. Deficient plants are small and their growth is retarded. Symptoms may vary between plant species. For example, in corn, sulfur deficiency shows up as interveinal chlorosis; in wheat, the whole plant becomes pale while the younger leaves are more chlorotic; in potatoes, spotting of leaves might occur.

Thiosulphates are sulfur fertilizers known to have urease and/or nitrification inhibiting properties. They are not only highly desirable fertilizers because they provide essential sulfur to plants, they also increase the nitrogen use efficiency (NUE) of nitrogen fertilizers used in conjunction with these thiosulphates, polysulfides and/or (bi) sulfites. This property stems from their chemical nature, in particular the fact that the sulfur in these compounds is not fully oxidized. The compounds are active as inhibitors, but also less stable, compared to e.g. sulphate fertilizers which do not exhibit any nitrification or urea inhibition. Only very recently, the first efforts to combine urea and nitrification inhibiting sulfur fertilizers such as thiosulphates, polysulfides and/or (bi) sulfites into a single, convenient, solid fertilizer formulation have been made, as described in WO2021/076458. Recent research efforts have also shown that it is important to use correct ratios of nitrogen to sulfur in order to obtain nitrification inhibiting effects in real-world applications.

WO2020/033575A1 describes various solid fertilizers comprising urea and thiosulphates, polysulfides and/or (bi) sulfites and methods of their production.

The present inventors have found that a disadvantage of known methods for the production of solid fertilizers comprising urea and a sulfur fertilizer such as thiosulphates, polysulfides and/or (bi) sulfites is that, opposed to relatively stable (fully oxidized) sulphate ions, these sulfur fertilizers are prone to partially decompose during processing, even in relatively mild conditions. This generates elemental sulfur and other sulfur-containing byproducts which, when integrated into an urea synthesis process, contaminate the urea synthesis equipment and adversely affect process performance, mechanical integrity and reliability. In particular, in a conventional urea synthesis facility wherein recovery of non-converted reagents is performed, an accumulation of elemental sulfur and other sulfur-containing byproducts occurs. This may lead to deterioration of equipment, decreased urea synthesis performance, decreased process efficiency, increased maintenance etc. There is thus a need to provide alternative or improved methods for the production of these solid fertilizers.

It is an object of the present invention to provide alternative or improved methods for the production of solid fertilizers comprising urea and a thiosulphate. It is a further object of the present invention to provide such methods wherein contamination of the urea synthesis plant with sulfur-containing compounds is reduced or avoided. It is a further object of the present invention to provide such methods wherein energy consumption is reduced.

### SUMMARY OF THE INVENTION

The present inventors have found that one or more objects of the invention is achieved by preparing a liquid urea-thiosulfate blend comprising 70-90 wt. % (by dry weight of the urea-thiosulfate blend) urea and 10-30 wt. % (by dry weight of the urea-thiosulfate blend) thiosulfate salt, and submitting it (optionally after concentrating) as a liquid to a solidification section wherein the liquid is converted to a particulate solid. Preferably, the liquid urea-thiosulfate is a melt prior to solidification. As will be shown in the appended examples, it was found that at these relative amounts of urea and thiosulfate salt a significant melting point depression occurs. This allows the process to be operated at significantly lower temperatures than those at which liquid urea compositions (such as urea melts) would regularly be processed (typically >128° C.), resulting in significant energy savings and associated cost savings, as well as limiting thiosulfate decomposition to elemental sulfur and other sulfur-containing byproducts. The limited amount of thiosulfate compared to urea is sufficient to cause significant melting point depression, as well as to result in significant nitrification and/or urease inhibition, but at the same time also ensures the absolute amount of decomposition products in the final urea-thiosulfate fertilizer is limited and thus a high quality fertilizer can be produced.

Hence, in a first aspect of the invention there is provided a method for the production of a solid urea-thiosulfate fertilizer, said method comprising the following steps:
(i) providing a first liquid composition comprising urea;
(ii) providing a second composition comprising a thiosulfate salt;
(iii) combining the composition provided in step (i) with the composition provided in step (ii) to obtain a liquid urea-thiosulfate blend comprising 70-90 wt. % (by dry weight of the urea-thiosulfate blend) urea and 10-30 wt. % (by dry weight of the urea-thiosulfate blend) thiosulfate salt;
(iv) optionally submitting the liquid urea-thiosulfate blend of step (iii) to a concentration step to obtain a concentrated liquid urea-thiosulfate blend; and
(v) feeding the liquid urea-thiosulfate blend of step (iii) and/or the concentrated liquid urea-thiosulfate blend of step (iv) as a liquid to a solidification section wherein the liquid is converted to a particulate solid, thereby obtaining the solid urea-thiosulfate fertilizer.

The present invention will now be described in more detail with reference to specific embodiments of the invention, given only by way of illustration, and with reference to the accompanying drawings.

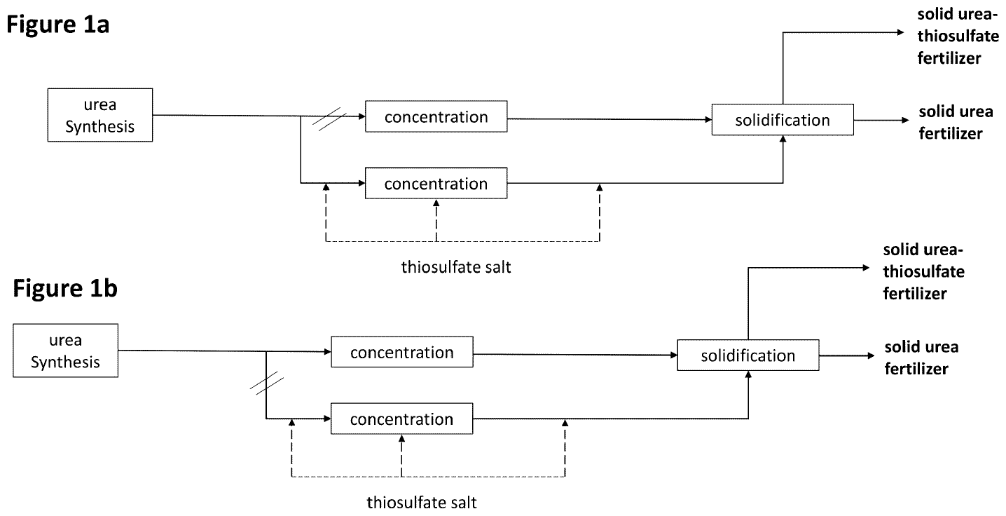

FIG. 1 is a schematic representation of an embodiment of the method for the production of a solid urea-thiosulfate fertilizer of the present invention, illustrating integration of the method of the present invention is with an urea production facility, FIG. 1a showing a first operational mode and FIG. 1b showing a second operational mode wherein an urea-thiosulfate fertilizer is produced in the first operational mode and a regular urea fertilizer is produced in the second operational mode, the first operational mode using an evaporator which is distinct from the evaporator used in the second operational mode, both modes using the same solidification apparatus.

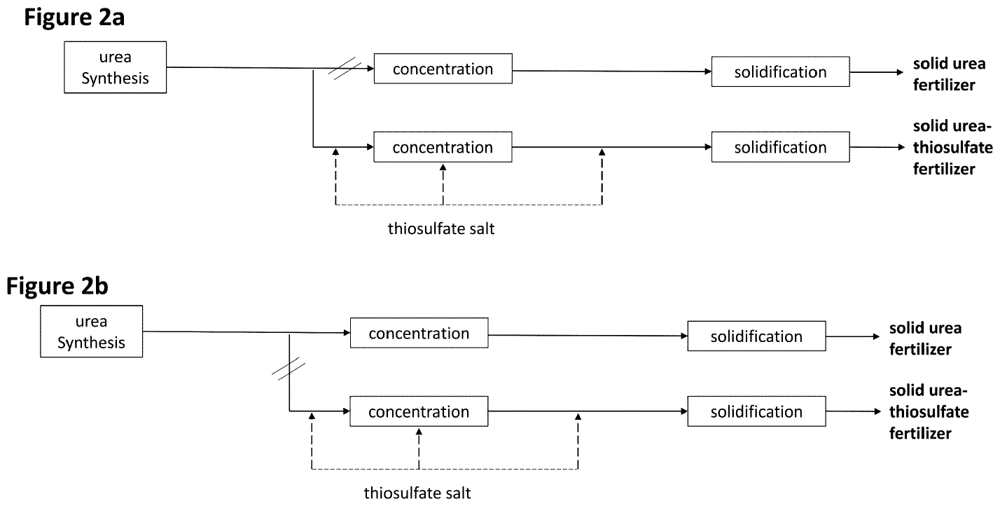

FIG. 2 is a schematic representation of an embodiment of the method for the production of a solid urea-thiosulfate fertilizer of the present invention, illustrating integration of the method of the present invention is with an urea production facility, FIG. 2a showing a first operational mode and FIG. 2b showing a second operational mode wherein an urea-thiosulfate fertilizer is produced in the first operational mode and a regular urea fertilizer is produced in the second operational mode, the first operational mode using an evaporator and solidification apparatus which are distinct from the evaporator and solidification apparatus used in the second operational mode.

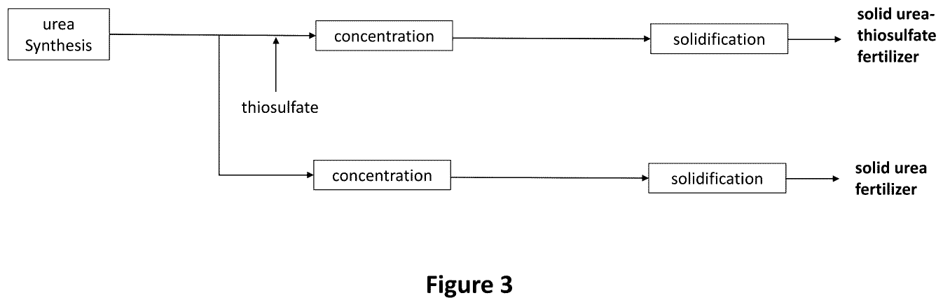

FIG. 3 is a schematic representation of an embodiment of the method for the production of a solid urea-thiosulfate fertilizer of the present invention, illustrating the simultaneous coproduction of urea fertilizer and urea-thiosulfate fertilizer in an urea production facility.

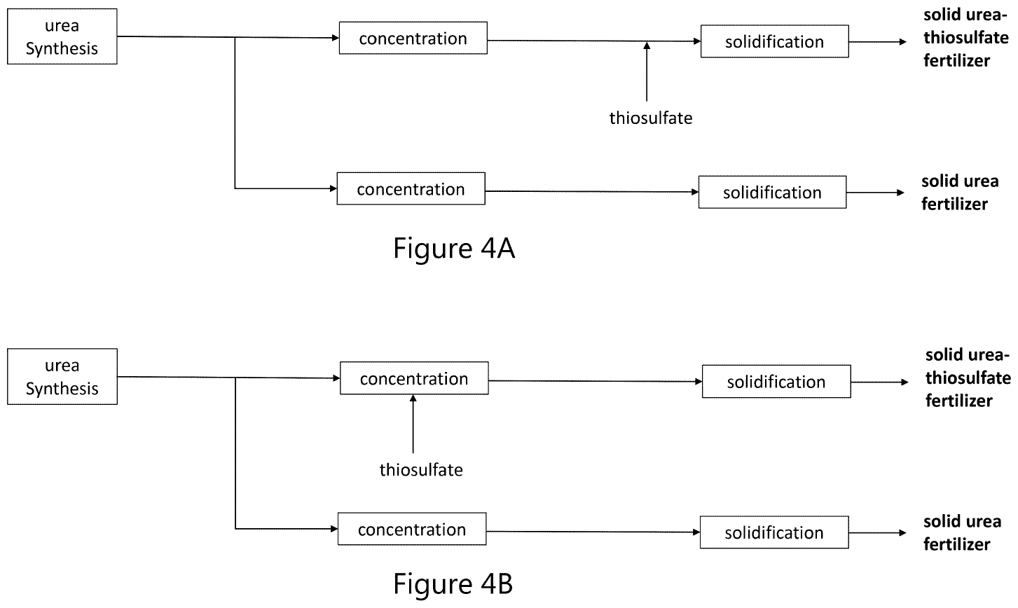

FIGS. 4a and 4b are a schematic representation of an embodiment of the method for the production of a solid urea-thiosulfate fertilizer of the present invention, illustrating the simultaneous coproduction of urea fertilizer and urea-thiosulfate fertilizer in an urea production facility.

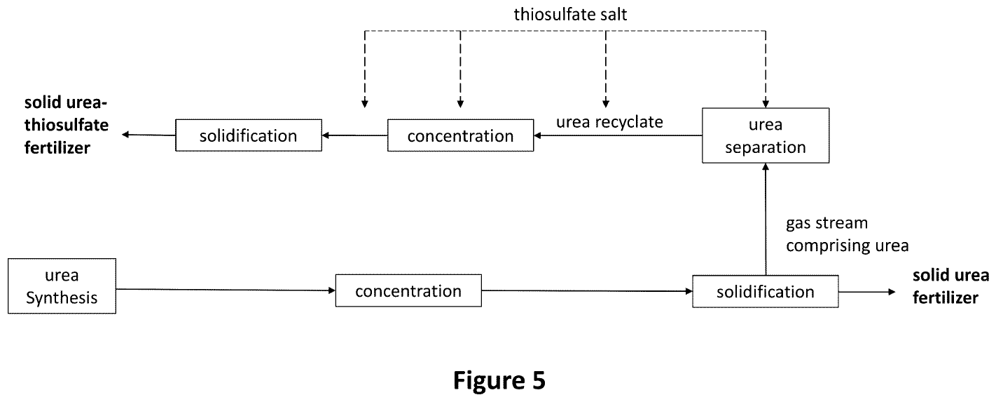

FIG. 5 is a schematic representation of an embodiment of the method for the production of a solid urea-thiosulfate fertilizer of the present invention, illustrating recovery of urea recyclate and using the recyclate for urea-thiosulfate fertilizer production.

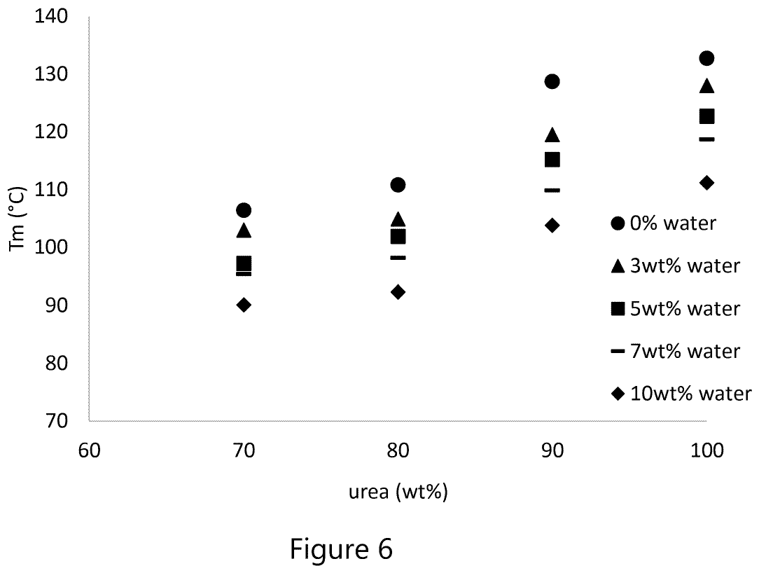

FIG. 6 illustrates the melting point of the various urea-ammonium thiosulfate mixtures of example 1.

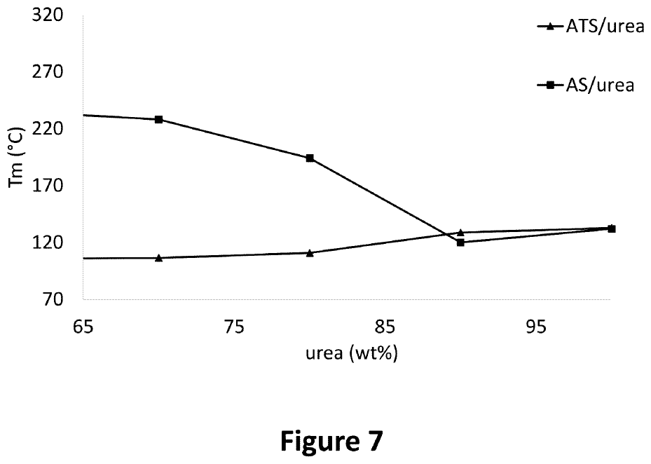

FIG. 7 illustrates the melting point of urea-ammonium thiosulfate mixtures compared to urea-ammonium sulfate mixtures as explained in example 1.

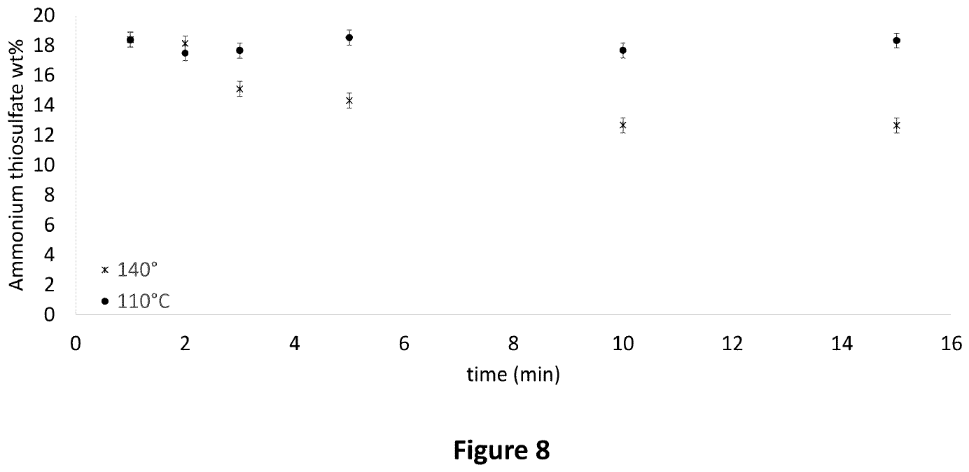

FIG. 8 illustrates the stability of the urea-ammonium thiosulfate at different temperatures as explained in example 2.

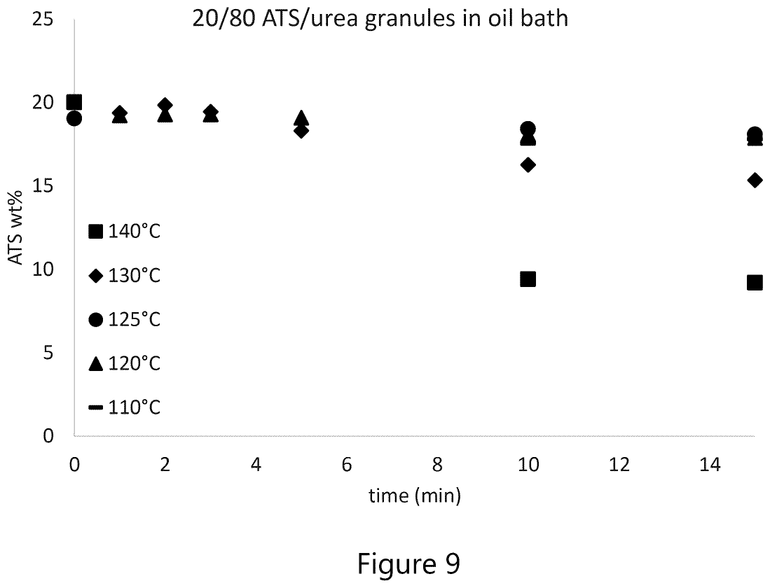

FIG. 9 illustrates the stability of the urea-ammonium thiosulfate in granular form at different temperatures as explained in example 2.

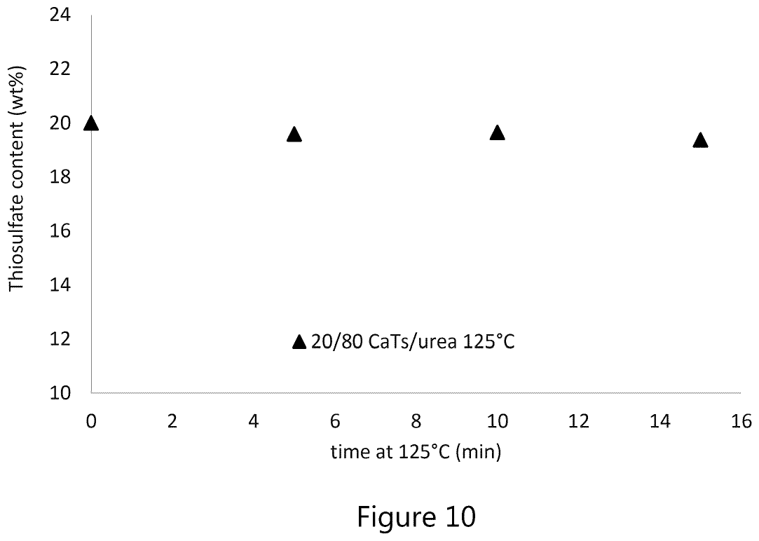

FIG. 10 illustrates the stability of the urea-calcium thiosulfate at a temperature of 125° C. as explained in example 2.

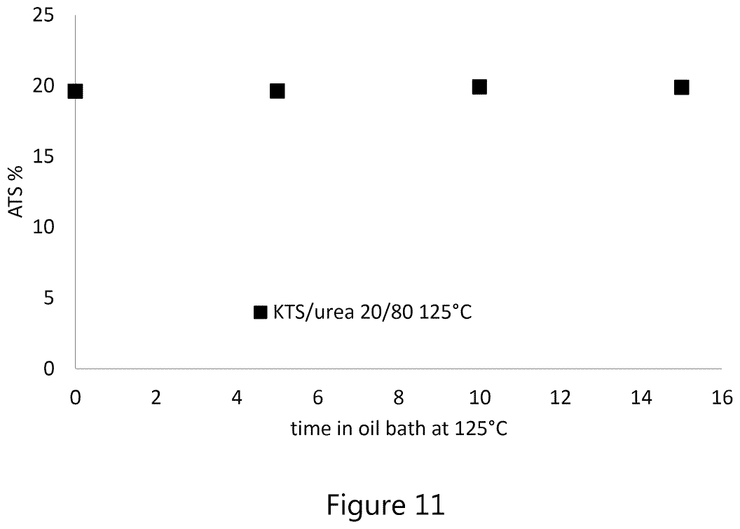

FIG. 11 illustrates the stability of the urea-potassium thiosulfate at a temperature of 125° C. as explained in example 2.

### DETAILED DESCRIPTION

The expression “comprise” and variations thereof, such as, “comprises” and “comprising” as used herein should be construed in an open, inclusive sense, meaning that the embodiment described includes the recited features, but that it does not exclude the presence of other features, as long as they do not render the embodiment unworkable.

The expressions “one embodiment”, “a particular embodiment”, “an embodiment” etc. as used herein should be construed to mean that a particular feature, structure or characteristic described in connection with the embodiment is included in at least one embodiment. Thus, the appearances of such expressions in various places throughout this specification do not necessarily all refer to the same embodiment. Furthermore, the particular features, structures, or characteristics may be combined in any suitable manner in one or more embodiments. For example, certain features of the disclosure which are described herein in the context of separate embodiments are also explicitly envisaged in combination in a single embodiment.

The singular forms “a,” “an,” and “the” as used herein should be construed to include plural referents unless the content clearly dictates otherwise. It should also be noted that the term “or” is generally employed in its broadest sense, that is as meaning “and/or” unless the content clearly dictates otherwise.

Whenever reference is made throughout this document to a compound which is a salt, this should be construed to include the anhydrous form as well as any solvates (in particular hydrates) of this compound, unless explicitly defined otherwise.

As used herein, the expression “wt. %” when used in the context of an ionic compound (such as a thiosulfate or a sulfate) refers to the amount of the compound inclusive of its counterion.

As used herein, the expression “particulate solid” is not particularly limited to the nature of the particulate solid and in particular includes granules, prills, pellets, pastilles and powders.

As used herein, the expression “fluidized bed granulator” includes vortex granulators.

The expression “thiosulphates” or “thiosulphate salts” as used herein refers to the salts of thiosulphuric acid, which consist of one or more cations combined with a thiosulphate (S₂O₃²⁻) anion.

The expression “elemental sulfur” as used herein refers to compounds consisting of sulfur in oxidation state 0, typically in the form of S₈ molecules.

The method of the present invention may be performed starting from various urea sources, such as solid urea which was dissolved to provide the first liquid composition of step (i); or solid urea which was melted to provide the first liquid composition of step (i); but may also be started from any one of different liquid urea streams which exist in a regular urea synthesis plant. In all the embodiments of the invention described herein, it is preferred that the combined amount of urea and water in the first liquid composition provided in step (i) is at least 95 wt. % (by total weight of the first liquid composition), preferably at least 98 wt. % (by total weight of the first liquid composition).

In preferred embodiments of the invention, the first liquid composition provided in step (i) is an urea melt comprising at least 95 wt. % (by total weight of the composition) urea, preferably comprising at least 96 wt. % (by total weight of the composition) urea. In this embodiment, the temperature of the urea melt provided in step (i) is preferably at least 128° C., preferably at least 130° C. In some embodiments the urea melt is substantially free of urea-formaldehyde, or substantially free of additives which lower the melting point of urea. As will be explained herein elsewhere in the context of specific embodiments, the present method can be optimally integrated in a regular urea synthesis plant such that the urea melt of these embodiments can be drawn from an urea production plant.

In alternative preferred embodiments of the invention, the first liquid composition provided in step (i) is an aqueous composition comprising at least 25 wt. % urea (by total weight of the first liquid composition) and at least 30 wt. % water (by total weight of the first liquid composition). In particular embodiments of the invention, the first liquid composition provided in step (i) is an aqueous composition comprising 25-45 wt. % (by total weight of the first liquid composition) urea and 55-75 wt. % (by total weight of the first liquid composition) water. As will be explained herein elsewhere in the context of specific embodiments, the present method can be optimally integrated in a regular urea production plant such that the first liquid composition comprising 25-45 wt. % (by total weight of the first liquid composition) urea and 55-75 wt. % (by total weight of the first liquid composition) water can be drawn from the urea dust scrubber installed on the solidification section of a urea synthesis plant. In other particular embodiments of the invention, the first liquid composition provided in step (i) is an aqueous composition comprising 65-95 wt. % (by total weight of the first liquid composition) urea and at least 5 wt. % (by total weight of the first liquid composition) water, preferably an aqueous composition comprising 65-75 wt. % (by total weight of the first liquid composition) urea and at least 20 wt. % (by total weight of the first liquid composition) water. As will be explained herein elsewhere in the context of specific embodiments, the present method can be optimally integrated in a regular urea production plant such that the first liquid composition comprising 65-95 wt. % (by total weight of the first liquid composition) urea and at least 5 wt. % (by total weight of the first liquid composition) water can be drawn from the urea synthesis section of a urea production plant before the evaporation section.

The thiosulfate salt provided in step (ii) is preferably selected from the group consisting of alkali metal thiosulphates, alkaline earth metal thiosulphates, iron thiosulphates, ammonium thiosulphates and combinations thereof, more preferably the thiosulfate salt is selected from the group consisting of calcium thiosulphates, magnesium thiosulphates, potassium thiosulphates, ammonium thiosulphates, manganese thiosulphates, iron thiosulphates, ammonium thiosulphates and combinations thereof, more preferably the thiosulfate salt is selected from the group consisting of potassium thiosulphates, calcium thiosulphates, ammonium thiosulphates and combinations thereof, most preferably thiosulfate salt is ammonium thiosulphate.

The composition comprising the thiosulfate salt of step (ii) may be provided as a solid, liquid or slurry. In some embodiments of the invention, the composition provided in step (ii) is provided in the form of a solid, preferably in the form of a solid comprising more than 90 wt. % (by total weight of the solid) of the thiosulfate salt, more preferably in the form of a solid comprising more than 90 wt. % (by total weight of the solid) of the thiosulfate salt and having a water content of less than 5 wt. % (by total weight of the solid), more preferably in the form of a solid comprising more than 90 wt. % (by total weight of the solid) of the thiosulfate salt and having a water content of less than 3 wt. % (by total weight of the solid).

In alternative, highly preferred embodiments of the invention, the composition provided in step (ii) is provided in the form of an aqueous solution of the thiosulfate salt, preferably in the form of an aqueous solution of the thiosulfate salt comprising at least 15 wt. % (by total weight of the aqueous solution provided in step (ii)) of the thiosulfate salt, preferably at least 30 wt. % (by total weight of the aqueous solution provided in step (ii)) of the thiosulfate salt. In preferred embodiments of the invention, the composition provided in step (ii) is provided in the form of an aqueous solution comprising:
ammonium thiosulfate in an amount resulting in a nitrogen content (as ammoniacal nitrogen) of more than 10 wt. % (by total weight of the aqueous solution provided in step (ii)) and a sulfur content of more than 24 wt. % (by total weight of the aqueous solution provided in step (ii)); or
potassium thiosulfate in an amount resulting in a potassium content (as K₂O) of more than 22 wt. % (by total weight of the aqueous solution provided in step (ii)) and a sulfur content of more than 15 wt. % (by total weight of the aqueous solution provided in step (ii)); or
calcium thiosulfate in an amount resulting in a calcium content of more than 5 wt. % (by total weight of the aqueous solution provided in step (ii)) and a sulfur content of more than 8 wt. % (by total weight of the aqueous solution provided in step (ii)); or
magnesium thiosulfate in an amount resulting in a magnesium content of more than 3 wt. % (by total weight of the aqueous solution provided in step (ii)) and a sulfur content of more than 8 wt. % (by total weight of the aqueous solution provided in step (ii));
   preferably
ammonium thiosulfate in an amount resulting in a nitrogen content (as ammoniacal nitrogen) of more than 10 wt. % (by total weight of the aqueous solution provided in step (ii)) and a sulfur content of more than 26 wt. % (by total weight of the aqueous solution provided in step (ii)).
   Advantageously, the method of the present invention allows the solid urea-thiosulfate fertilizer to be prepared from commercially available liquid fertilizers, for example starting from a liquid thiosulfate product which is produced and sold as such (e.g. Thio-Sul®, KTS®, CaTs or MagThio® available from Tessenderlo Group NV or its subsidiaries). Advantageously, these products which already contain high thiosulfate concentrations close to the solubility limit, can be used to conveniently add thiosulfate to the composition of step (i) with minimal introduction of water, which needs to be evaporated before solidification.

In highly preferred embodiments of the invention, step (iv) is applied, preferably step (iv) is applied such that the concentrated liquid urea-thiosulfate blend obtained in step (iv) has a water content of less than 5 wt. % (by total weight of the concentrated blend), preferably less than 4 wt. % (by total weight of the concentrated blend). The application of a concentration step (iv) is particularly preferred in case the thiosulfate salt provided in step (ii) is provided in the form of an aqueous solution of thiosulfate salt.

In accordance with preferred embodiments of the invention, concentration step (iv) is performed by evaporation. Concentration step (iv) may be performed as a single or multi-stage evaporation. The type of evaporator(s) employed is not particularly limiting, and may be for example selected from the group consisting of falling-film evaporators, rising film evaporators, thin-film evaporators, wiped film evaporators, short path evaporators, forced circulation evaporators, plate evaporators, plate and frame evaporators, shell-and-tube evaporators and combinations thereof. The evaporation of step (iv) is preferably performed using falling-film evaporation, wiped-film evaporation and combinations thereof. The evaporator(s) may be operated in known modes such as single or multiple pass, multiple-effect, employing thermal vapor recompression, employing mechanical vapor recompression, etc. In highly preferred embodiments of the invention, the evaporation of step (iv) is performed at a temperature of less than 128° C., preferably a temperature of equal to or less than 125° C., most preferably a temperature within the range of 100-125° C. As is shown in the appended examples, the present inventors have found that the urea-thiosulfate ratios prescribed by the present method result in a significant melting point reduction, which allows the evaporation of step (iv) to be performed at significantly reduced temperatures resulting in energy saving as well as reduced thiosulfate decomposition. In some embodiments the evaporation of step (iv) includes an evaporation stage performed at a pressure of more than 0.8 atm, preferably more than 0.9 atm, more preferably more than 0.95 atm and a temperature of less than 128° C., preferably a temperature of equal to or less than 125° C., most preferably a temperature within the range of 100-125° C., for example a pressure of more than 0.8 atm, preferably more than 0.9 atm, more preferably more than 0.95 atm and a temperature within the range of 100-125° C. In some embodiments the evaporation of step (iv) includes an evaporation stage performed at a pressure of less than 0.8 atm, preferably less than 0.7 atm, more preferably less than 0.5 atm and a temperature of less than 128° C., preferably a temperature of equal to or less than 125° C., most preferably a temperature within the range of 100-125° C., for example a pressure of less than 0.8 atm, preferably less than 0.7 atm, more preferably less than 0.5 atm and a temperature within the range of 100-125° C. The advantage of decreasing the vacuum pressure using these temperatures is that it can facilitate reduction of the moisture content (i.e. water content) of the liquid urea-thiosulfate blend. It is preferred that the temperature of the concentrated liquid urea-thiosulfate blend exiting the evaporator is less than 128° C., preferably a temperature of equal to or less than 125° C., most preferably a temperature within the range of 100-125° C. In a highly preferred embodiment of the invention, step (iii) is performed before the concentration step (iv). Similarly, it is preferred that the liquid urea-thiosulfate blend of step (iii) and/or the concentrated liquid urea-thiosulfate blend of step (iv) is fed to the solidification section at a temperature of less than 128° C., preferably a temperature of equal to or less than 125° C., most preferably a temperature within the range of 100-125° C. The methods according to the embodiments specifying maximal temperatures described herein are enabled thanks to the surprising and significant melting point depression found for the urea-thiosulfate blends specified in the present methods, and result in a low amount of thiosulfate degradation as well as increased energy efficiency.

The solidification section of step (v) preferably comprises a solidification apparatus selected from a prilling tower, a pelletizer, a fluidized bed granulator, a drum granulator, a falling curtain granulator, a spray dryer, a pan granulator, an extruder, a rotoformer, an oil priller and a compactor. More preferably, the solidification apparatus comprised in the solidification section of step (v) is selected from a prill tower, a rotoformer, a drum granulator and a fluidized bed granulator. In general, it is highly preferred that the liquid urea-thiosulfate blend of step (iii) and/or the concentrated liquid urea-thiosulfate blend of step (iv) which is fed to the solidification apparatus has a water content of less than 5 wt. % (by total weight of the liquid fed to the solidification apparatus), preferably less than 4 wt. % (by total weight of the liquid fed to the solidification apparatus). In case the solidification apparatus comprised in the solidification section of step (v) is a prill tower or a rotoformer, it is preferred that the liquid urea-thiosulfate blend of step (iii) and/or the concentrated liquid urea-thiosulfate blend of step (iv) which is fed to the solidification apparatus has a water content of less than 1 wt. % (by total weight of the liquid fed to the solidification apparatus), preferably less than 0.5 wt. % (by total weight of the liquid fed to the solidification apparatus).

Step (iv) is preferably performed, in particular in case the second composition provided in step (ii) is provided in the form of an aqueous solution of thiosulfate salt as described herein elsewhere. Hence, in general step (iv) preferably comprises concentrating the liquid urea-thiosulfate blend of step (iii) to obtain a concentrated liquid urea-thiosulfate blend having a water content of less than 5 wt. % (by total weight of the concentrated liquid urea-thiosulfate blend), preferably less than 4 wt. % (by total weight of the concentrated liquid urea-thiosulfate blend) which is sufficient for most solidification apparatuses. In case the solidification section comprises a solidification apparatus selected from a prill tower or a rotoformer, step (iv) preferably comprises concentrating the liquid urea-thiosulfate blend of step (iii) to obtain a concentrated liquid urea-thiosulfate blend having a water content of less than 1 wt. % (by total weight of the concentrated liquid urea-thiosulfate blend), preferably less than 0.5 wt. % (by total weight of the concentrated liquid urea-thiosulfate blend). In case the solidification section comprises a solidification apparatus which is a drum granulator and/or a fluidized bed granulator, higher moisture levels are tolerated, such that it is generally sufficient if step (iv) comprises concentrating the liquid urea-thiosulfate blend of step (iii) to obtain a concentrated liquid urea-thiosulfate blend having a water content of less than 5 wt. % (by total weight of the concentrated liquid urea-thiosulfate blend), preferably less than 4 wt. % (by total weight of the concentrated liquid urea-thiosulfate blend).

In accordance with highly preferred embodiments of the invention, step (iii) is performed outside the solidification section of step (v), preferably before the concentration step (iv). In this way the process optimally exploits the energy efficiency gains provided by the decreased melting point of the urea-thiosulfate blend. Furthermore, in case the thiosulfate is added in the form of an aqueous solution of thiosulfate, as is explained herein elsewhere to be preferred, the water brought by the thiosulfate solution can be at least partially removed in the concentration step (iv), preferably the evaporation of step (iv).

In some embodiments of the invention, the solid urea-thiosulfate fertilizer of step (v) is substantially free of urea-formaldehyde.

In highly preferred embodiments, the method of the present invention is integrated with an urea production facility. In such embodiments, step (i) comprises the following steps:
(i)a synthesizing urea from ammonia and carbon dioxide thereby obtaining a liquid aqueous composition comprising urea;
(i)b optionally concentrating the aqueous composition obtained in step (i) to obtain a liquid urea melt comprising more than 95 wt. % (by total weight of the melt) urea
(i)c optionally submitting the urea melt of step (i)b to a solidification step in a solidification section wherein the melt is converted to a particulate solid, thereby obtaining a solid urea fertilizer, and optionally recovering from the solidification section a gas stream comprising urea, such as urea dust; and
(i)d optionally separating urea from the gas stream recovered from the solidification section, thereby obtaining an urea recyclate which is preferably a liquid aqueous composition comprising urea, more preferably the urea recyclate is an aqueous composition comprising 25-45 wt. % urea (by total weight of the composition) and 55-75 wt. % water;
   wherein the first liquid composition comprising urea which is provided in step (i) is the liquid aqueous composition obtained in step (i)a; the liquid urea melt obtained in step (i)b and/or the urea recyclate obtained in step (i)d.

The ammonia and carbon dioxide utilized in step (i)a for the synthesis of urea can originate from any source, such as the Haber-Bosch process, electrochemical production, bio-based production (e.g. fermentation by bacteria, yeast or other micro-organisms), carbon capture from gaseous process streams or the atmosphere, etc.

In some embodiments of the invention, the method of the present invention is integrated with a urea production facility as described herein earlier and all of the liquid aqueous composition obtained in step (i)a is provided in step (i) for the production of a urea-thiosulfate based fertilizer, and steps (i)b, (i)c and (i)d are not performed. This embodiment corresponds to using all the urea produced by a urea synthesis plant for urea-thiosulfate fertilizer production, wherein the thiosulfate is added to the liquid aqueous urea composition obtained in step (i)a. In alternative embodiments of the invention, the method of the present invention is integrated with a urea production facility as described herein earlier, step (i)b is performed and all of the liquid urea melt obtained in step (i)b is provided in step (i) for the production of a urea-thiosulfate based fertilizer, and steps (i)c and (i)d are not performed. This embodiment corresponds to using all the urea produced by a urea synthesis plant for urea-thiosulfate fertilizer production, wherein the thiosulfate is added to the liquid urea melt obtained in step (i)b.

In some embodiments of the invention, the method of the present invention is integrated with a urea production facility as described herein earlier wherein the urea production facility is first operated in a first operational mode wherein
part or all of the liquid aqueous composition obtained in step (i)a is provided in step (i) for the production of a urea-thiosulfate based fertilizer, and steps (i)b, (i)c and (i)d are not performed; or
part or all of the liquid urea melt obtained in step (i)b is provided in step (i) for the production of a urea-thiosulfate based fertilizer, and steps (i)c and (i)d are not performed;
   and wherein after completion of the production of urea-thiosulfate based fertilizer according to steps (i)-(v), the urea production facility is subsequently operated in a second operational mode wherein
all of the liquid aqueous composition obtained in step (i)a is used for the production of a particulate solid which comprises more than 95 wt. % (by dry weight of the particulate solid) urea, preferably more than 98 wt. % (by dry weight of the particulate solid) urea according to steps (i)b-(i)c.
   In some embodiments of the invention, the concentration steps (iv) is performed in an evaporator, and the second operational mode employs the same evaporator in step (i)b as the evaporator which is used in step (iv) of the first operational mode, such that the evaporator of step (iv) is used alternatingly for the production of urea-thiosulfate based fertilizer and of urea fertilizer.

In alternative, highly preferred embodiments of the invention, the concentration step (iv) is performed in an evaporator and the second operational mode employs an evaporator in step (i)b which is distinct from the evaporator which is used in step (iv) of the first operational mode. In this way, evaporator condensate can be recycled to step (i)a when the second operational mode is used without risk of contaminating the urea synthesis section with elemental sulfur or other sulfur containing byproducts. Hence, it is preferred that the method further comprises recycling at least part of the condensate from the evaporator of step (i)b to step (i)a, while not recycling any of the condensate from the evaporator of step (iv) to step (i)a. In some embodiments of the invention, the concentration step (iv) is performed in an evaporator and the second operational mode employs an evaporator in step (i)b which is distinct from the evaporator which is used in step (iv) of the first operational mode and the first operational mode is performed when the evaporator used in step (i)b is undergoing maintenance. In some embodiments of the invention, the second operational mode employs the same solidification apparatus in step (i)c as the solidification apparatus which is used in step (v) of the first operational mode, such that the solidification apparatus of step (v) is used alternatingly for the production of urea-thiosulfate based fertilizer and of urea fertilizer. In alternative, embodiments, the second operational mode employs a solidification apparatus in step (i)c which is distinct from the solidification apparatus which is used in step (v) of the first operational mode.

FIGS. 1a and 1b illustrate an embodiment of the invention wherein the method of the present invention is integrated with an urea production facility, wherein step (i) comprises the following steps:
(i)a synthesizing urea from ammonia and carbon dioxide thereby obtaining a liquid aqueous composition comprising urea;
(i)b optionally concentrating the aqueous composition obtained in step (i) to obtain a liquid urea melt comprising more than 95 wt. % (by total weight of the melt) urea
(i)c optionally submitting the urea melt of step (i)b to a solidification step in a solidification section wherein the melt is converted to a particulate solid, thereby obtaining a solid urea fertilizer, and optionally recovering from the solidification section a gas stream comprising urea, such as urea dust; and
(i)d optionally separating urea from the gas stream recovered from the solidification section, thereby obtaining an urea recyclate which is preferably a liquid aqueous composition comprising urea, more preferably the urea recyclate is an aqueous composition comprising 25-45 wt. % urea (by total weight of the composition) and 55-75 wt. % water;
   wherein the first liquid composition comprising urea which is provided in step (i) is the liquid aqueous composition obtained in step (i)a; and

wherein the urea production facility is first operated in a first operational mode wherein
part or all of the liquid aqueous composition obtained in step (i)a is provided in step (i) for the production of a urea-thiosulfate based fertilizer, and steps (i)b, (i)c and (i)d are not performed;
   and wherein after completion of the production of urea-thiosulfate based fertilizer according to steps (i)-(v), the urea production facility is subsequently operated in a second operational mode wherein
part or all of the liquid aqueous composition obtained in step (i)a is used for the production of a particulate solid which comprises more than 95 wt. % (by dry weight of the particulate solid) urea, preferably more than 98 wt. % (by dry weight of the particulate solid) urea according to steps (i)b-(i)c;
   wherein the concentration step (iv) is performed in an evaporator and the second operational mode employs an evaporator in step (i)b which is distinct from the evaporator which is used in step (iv) of the first operational mode; and
   wherein the second operational mode employs the same solidification apparatus in step (i)c as the solidification apparatus which is used in step (v) of the first operational mode, such that the solidification apparatus of step (v) is used alternatingly for the production of urea-thiosulfate based fertilizer and of urea fertilizer. FIG. 1a shows the first operational mode, while FIG. 1b shows the second operational mode. The solidification sections (i)c and (v) comprise a solidification apparatus as described herein earlier. The dotted line in FIG. 1a and FIG. 1b illustrates a non-limiting number of example points of the process where the thiosulfate salt provided in step (ii) can be introduced either singly or in combination.

FIGS. 2a and 2b illustrate an embodiment of the invention wherein the method of the present invention is integrated with an urea production facility, wherein step (i) comprises the following steps:
(i)a synthesizing urea from ammonia and carbon dioxide thereby obtaining a liquid aqueous composition comprising urea;
(i)b optionally concentrating the aqueous composition obtained in step (i) to obtain a liquid urea melt comprising more than 95 wt. % (by total weight of the melt) urea
(i)c optionally submitting the urea melt of step (i)b to a solidification step in a solidification section wherein the melt is converted to a particulate solid, thereby obtaining a solid urea fertilizer, and optionally recovering from the solidification section a gas stream comprising urea, such as urea dust; and
(i)d optionally separating urea from the gas stream recovered from the solidification section, thereby obtaining an urea recyclate which is preferably a liquid aqueous composition comprising urea, more preferably the urea recyclate is an aqueous composition comprising 25-45 wt. % urea (by total weight of the composition) and 55-75 wt. % water;
   wherein the first liquid composition comprising urea which is provided in step (i) is the liquid aqueous composition obtained in step (i)a; and

wherein the urea production facility is first operated in a first operational mode wherein
part or all of the liquid aqueous composition obtained in step (i)a is provided in step (i) for the production of a urea-thiosulfate based fertilizer, and steps (i)b, (i)c and (i)d are not performed; and wherein after completion of the production of urea-thiosulfate based fertilizer according to steps (i)-(v), the urea production facility is subsequently operated in a second operational mode wherein
part or all of the liquid aqueous composition obtained in step (i)a is used for the production of a particulate solid which comprises more than 95 wt. % (by dry weight of the particulate solid) urea, preferably more than 98 wt. % (by dry weight of the particulate solid) urea according to steps (i)b-(i)c;
   wherein the concentration step (iv) is performed in an evaporator and the second operational mode employs an evaporator in step (i)b which is distinct from the evaporator which is used in step (iv) of the first operational mode; and
   wherein the second operational mode employs a solidification apparatus in step (i)c which is distinct from the solidification apparatus which is used in step (v) of the first operational mode. FIG. 2a shows the first operational mode, while FIG. 2b shows the second operational mode. The dotted line in FIG. 2a and FIG. 2b illustrates a non-limiting number of example points of the process where the thiosulfate salt provided in step (ii) can be introduced either singly or in combination. The solidification sections (i)c and (v) comprise a solidification apparatus as described herein earlier.

In some embodiments of the invention, the method is for the coproduction of urea fertilizer and urea-thiosulfate fertilizer in an urea production facility. In such embodiments, step (i) comprises the following steps:
(i)a synthesizing urea from ammonia and carbon dioxide thereby obtaining a liquid aqueous composition comprising urea;
(i)b concentrating the aqueous composition obtained in step (i) to obtain a liquid urea melt comprising more than 95 wt. % (by total weight of the melt) urea
(i)c submitting the urea melt of step (i)b to a solidification step in a solidification section wherein the melt is converted to a particulate solid, thereby obtaining a solid urea fertilizer, and optionally recovering from the solidification section a gas stream comprising urea, such as urea dust; and
(i)d optionally separating urea from the gas stream recovered from the solidification section, thereby obtaining an urea recyclate which is preferably a liquid aqueous composition comprising urea, more preferably the urea recyclate is an aqueous composition comprising 25-45 wt. % urea (by total weight of the composition) and 55-75 wt. % water;
   wherein the first liquid composition comprising urea which is provided in step (i) is part of the liquid aqueous composition obtained in step (i)a; part of the liquid urea melt obtained in step (i)b and/or part or all of the urea recyclate obtained in step (i)d. In preferred embodiments, the first liquid composition comprising urea which is provided in step (i) is part or all of the urea recyclate obtained in step (i)d.
   In some embodiments of the invention the liquid aqueous composition obtained in step (i)a is split into a portion which is provided in step (i) for the production of a urea-thiosulfate based fertilizer and another portion which is submitted to step (i)b and (i)c for the production of a solid urea fertilizer, wherein optionally step (i)d is performed.
   In some embodiments of the invention the liquid urea melt obtained in step (i)b is split into a portion which is provided in step (i) for the production of a urea-thiosulfate based fertilizer and another portion which is submitted to step (i)c for the production of a solid urea fertilizer, wherein optionally step (i)d is performed.

In highly preferred embodiments of the invention, the simultaneous coproduction of urea fertilizer and urea-thiosulfate fertilizer in an urea production facility as described herein is provided wherein steps (i)b and (i)c are performed simultaneously with the production of urea-thiosulfate based fertilizer according to steps (i)-(v), wherein
step (iv) comprises concentrating the liquid urea-thiosulfate blend in a first evaporator;
step (i)b comprises concentrating the liquid composition of step (i)b in a second evaporator, wherein the second evaporator employed in step (i)b is a distinct apparatus from the first evaporator employed in step (iv); and
preferably step (i)c is performed employing a second solidification apparatus distinct from the solidification apparatus of step (v).
   In such embodiments, the process of the present invention has the particular advantage that, since the sulfur-containing streams employ a dedicated evaporator, solid nitrogen-sulfur fertilizer as described herein can be produced without contamination of the urea synthesis plant by sulfur compounds of the present invention, elemental sulfur and/or other sulfur containing byproducts, and in particular without build-up in the urea synthesis plant of sulfur compounds of the present invention, elemental sulfur and/or other sulfur containing byproducts. Hence, a portion of the urea originating from the synthesis plant of step (i)a is used for the production of the urea solid urea fertilizer of step (i)c; while simultaneously another portion of the urea originating from the synthesis plant of step (i)a is used for the production of the thiosulfate-urea based fertilizer of step (v). The solidification sections (i)c and (v) comprise a solidification apparatus as described herein earlier.

FIG. 3 illustrates an embodiment of the method of the present invention which is for the simultaneous coproduction of urea fertilizer and urea-thiosulfate fertilizer in an urea production facility, wherein step (i) comprises the following steps:
(i)a synthesizing urea from ammonia and carbon dioxide thereby obtaining a liquid aqueous composition comprising urea;
(i)b concentrating the aqueous composition obtained in step (i) to obtain a liquid urea melt comprising more than 95 wt. % (by total weight of the melt) urea
(i)c submitting the urea melt of step (i)b to a solidification step in a solidification section wherein the melt is converted to a particulate solid, thereby obtaining a solid urea fertilizer, and optionally recovering from the solidification section a gas stream comprising urea, such as urea dust; and
(i)d optionally separating urea from the gas stream recovered from the solidification section, thereby obtaining an urea recyclate which is preferably a liquid aqueous composition comprising urea, more preferably the urea recyclate is an aqueous composition comprising 25-45 wt. % urea (by total weight of the composition) and 55-75 wt. % water;
   wherein the first liquid composition comprising urea which is provided in step (i) is part of the liquid aqueous composition obtained in step (i)a. The solidification sections (i)c and (v) comprise a solidification apparatus as described herein earlier. The line in FIG. 3 illustrates a non-limiting example point of the process where the thiosulfate salt provided in step (ii) can be introduced.

FIGS. 4a and 4b illustrate an embodiment of the method of the present invention which is for the simultaneous coproduction of urea fertilizer and urea-thiosulfate fertilizer in an urea production facility, wherein step (i) comprises the following steps:
(i)a synthesizing urea from ammonia and carbon dioxide thereby obtaining a liquid aqueous composition comprising urea;
(i)b concentrating the aqueous composition obtained in step (i) to obtain a liquid urea melt comprising more than 95 wt. % (by total weight of the melt) urea
(i)c submitting the urea melt of step (i)b to a solidification step in a solidification section wherein the melt is converted to a particulate solid, thereby obtaining a solid urea fertilizer, and optionally recovering from the solidification section a gas stream comprising urea, such as urea dust; and
(i)d optionally separating urea from the gas stream recovered from the solidification section, thereby obtaining an urea recyclate which is preferably a liquid aqueous composition comprising urea, more preferably the urea recyclate is an aqueous composition comprising 25-45 wt. % urea (by total weight of the composition) and 55-75 wt. % water;
   wherein the first liquid composition comprising urea which is provided in step (i) is part of the liquid urea melt obtained in step (i)b. The solidification sections (i)c and (v) comprise a solidification apparatus as described herein earlier. The line in FIGS. 4a and 4b illustrates a non-limiting example point of the process where the thiosulfate salt provided in step (ii) can be introduced.

In the context of the present invention it is highly preferred that urea fertilizer obtained in step (i)c is substantially free of ammonium thiosulfate, preferably wherein the particulate solid obtained in step (i)c comprises more than 98 wt. % (by dry weight of the urea fertilizer) urea. Typically some byproducts will be present in small amounts, such as biuret. The amount of biuret is preferably less than 2 wt. % (by dry weight of the urea fertilizer), more preferably less than 1.5 wt. % (by dry weight of the urea fertilizer), more preferably less than 1.2 wt. % (by dry weight of the urea fertilizer), such as less than 1.0 wt. % (by dry weight of the urea fertilizer). In some embodiments the amount of biuret is less than 0.5 wt. % (by dry weight of the urea fertilizer) such that the urea is suitable for foliar use. Typically 0.3-0.4 wt. % (by dry weight of the urea fertilizer) formaldehyde will be present as anticaking agent, along with ammonia (typically <500 ppm (by dry weight of the urea fertilizer)) and other impurities. Hence, the present invention provides methods of retrofitting existing urea production facilities to co-produce solid urea-thiosulfate fertilizer.

In some embodiments of the invention, the solid urea-thiosulfate fertilizer of step (v) and the solid urea fertilizer obtained in step (i)c is substantially free of urea-formaldehyde.

In preferred embodiments of the invention step (i)a comprises at least partially separating the urea from non-converted reagents such as ammonia, carbon dioxide and ammonium carbamate in one or more recovery sections, thereby obtaining a liquid aqueous composition comprising 65-95 wt. % (by total weight of the composition) urea and at least 5 wt. % (by total weight of the composition) water, preferably obtaining a liquid aqueous composition comprising 65-75 wt. % (by total weight of the composition) urea and at least 20 wt. % (by total weight of the composition) water. The composition obtained from step (i)a and submitted to step (ii)b preferably comprises low amounts of byproducts and additives, such as less than 10 wt. % (by total weight of the composition) of compounds other than urea and water, preferably less than 5 wt. % (by total weight of the composition) of compounds other than urea and water. Common byproducts present in the aqueous urea composition obtained in step (i)a are biuret, and unconverted reagents such as ammonia, CO₂ and/or ammonium carbamate. In accordance with highly preferred embodiments of the invention, step (i)a comprises at least partially separating the urea from non-converted reagents such as ammonia, carbon dioxide and ammonium carbamate, thereby obtaining a liquid aqueous composition comprising 65-95 wt. % (by total weight of the composition) urea and at least 5 wt. % (by total weight of the composition) water, preferably obtaining a liquid aqueous composition comprising 65-75 wt. % (by total weight of the composition) urea and at least 20 wt. % (by total weight of the composition) water, and further comprises recycling at least part of the non-converted reagents to the urea synthesis reaction. In such embodiments, the process of the present invention has the particular advantage that urea-thiosulfate fertilizer as described herein can be produced without contamination of the urea synthesis plant by thiosulfate, elemental sulfur and/or other sulfur containing byproducts, and in particular without build-up in the urea synthesis plant of thiosulfate, elemental sulfur and/or other sulfur containing byproducts.

In highly preferred embodiments of the invention, concentration step (i)b is performed by evaporation. Step (i)b may be performed as a single or multi-stage evaporation. The type of evaporator(s) employed is not particularly limiting, and may for example be selected from the group consisting of falling-film evaporators, rising film evaporators, thin-film evaporators, wiped film evaporators, short path evaporators, forced circulation evaporators, shell-and-tube evaporators, plate evaporators, plate and frame evaporators and combinations thereof. The evaporation is preferably performed using falling-film evaporation. The evaporator(s) may be operated in known modes such as single or multiple pass, multiple-effect, employing thermal vapor recompression, employing mechanical vapor recompression, etc. In practice, step (i)b typically comprises at least two evaporation stages, each performed at a different temperature-pressure combination, in order to optimize process efficiency while avoiding solidification of urea in the evaporator (which would lead to process failure and plant shutdown). A typical scheme comprises a first evaporation step at 130° C. and a first reduced pressure (typically below 500 mbar), followed by a second evaporation step at 137-140° C. at a second reduced pressure which is lower than the first reduced pressure (typically below 100 mbar). In general it is preferred that the temperature of the urea melt is more than 128° C. when exiting the evaporator, in order to avoid solidification of urea before the process stream enters the solidification point (e.g. when passing cold spots). In accordance with highly preferred embodiments of the invention, the methods for the simultaneous coproduction of urea fertilizer and urea-thiosulfate fertilizer in an urea production facility are provided wherein step (i)b comprises concentrating the aqueous composition obtained in step (i)a by evaporation and further comprises recycling at least part of the condensate to the urea synthesis reaction. In such embodiments, the process of the present invention has the particular advantage that since no thiosulfate is present in this stage of the process, solid urea-thiosulfate fertilizer as described herein can be produced without contamination of the urea synthesis plant by thiosulfate, elemental sulfur and/or other sulfur containing byproducts, and in particular without build-up in the urea synthesis plant of thiosulfate, elemental sulfur and/or other sulfur containing byproducts.

It is particularly preferred that the embodiments of the invention described in the previous paragraphs are combined such that recycling of non-converted reagents from a recovery section during step (i)a and recycling of evaporator condensate during step (i)b both occur, as is typically the case in an integrated urea synthesis plant.

The extent of evaporation required in step (i)b is largely dependent on the maximum moisture content accepted by the solidification process employed by the solidification section of step (i)c, and it is within the routine capabilities of the skilled person, based on the present disclosure, to optimize this. In some embodiments of the invention, step (i)b comprises concentrating the aqueous composition obtained in step (i)a to obtain a liquid urea melt having a water content of less than 1 wt. % (by total weight of the liquid urea melt), preferably less than 0.5 wt. %. In such embodiments, step (i)b preferably comprises a two-step evaporation process starting from a liquid aqueous composition comprising 65-75 wt. % (by total weight of the composition) urea and at least 20 wt. % (by total weight of the composition) water which is concentrated to 93-97 wt. % (by total weight of the composition) urea in a first evaporation stage using a first temperature-pressure combination, and subsequently concentrated to more than 99 wt. % (by total weight of the composition) urea in a second evaporation stage using a second temperature-pressure combination.

The solidification section of step (i)c preferably comprises a solidification apparatus selected from a prilling tower, a pelletizer, a fluidized bed granulator, a drum granulator, a falling curtain granulator, a spray dryer, a pan granulator, an extruder, a rotoformer, an oil priller and a compactor. More preferably, the solidification section of step (i)c comprises a solidification apparatus selected from a prill tower, a rotoformer, a drum granulator and a fluidized bed granulator. In case the solidification apparatus is a prill tower or a rotoformer, step (i)b preferably comprises concentrating the aqueous composition obtained in step (i)a to obtain a liquid urea melt having a water content of less than 1 wt. % (by total weight of the liquid urea melt), preferably less than 0.5 wt. % (by total weight of the liquid urea melt). This is preferably done using the two-step evaporation process described in the previous paragraph. In case the solidification apparatus is a drum granulator and/or a fluidized bed granulator, higher moisture levels are tolerated, such that the water content of less than 5 wt. % (by total weight of the composition) prescribed by step (i)b is generally sufficient. Preferably, step (i)b comprises concentrating the aqueous composition obtained in step (i) to obtain a liquid urea melt having a water content of less than 4 wt. % (by total weight of the composition), preferably less than 3 wt. % (by total weight of the composition).

In some embodiments of the invention, step (i)d is performed and the first liquid composition comprising urea which is provided in step (i) is part or all of the urea recyclate obtained in step (i)d. FIG. 5 illustrates an embodiment of the method of the present invention which is for the coproduction of urea fertilizer and urea-thiosulfate fertilizer in an urea production facility, wherein step (i) comprises the following steps:
(i)a synthesizing urea from ammonia and carbon dioxide thereby obtaining a liquid aqueous composition comprising urea;
(i)b concentrating the aqueous composition obtained in step (i) to obtain a liquid urea melt comprising more than 95 wt. % (by total weight of the melt) urea
(i)c submitting the urea melt of step (i)b to a solidification step in a solidification section wherein the melt is converted to a particulate solid, thereby obtaining a solid urea fertilizer, and optionally recovering from the solidification section a gas stream comprising urea, such as urea dust; and
(i)d separating urea from the gas stream recovered from the solidification section, thereby obtaining an urea recyclate which is a liquid aqueous composition comprising urea, preferably the urea recyclate is an aqueous composition comprising 30-50 wt. % urea (by total weight of the composition) and at least 40 wt. % water;
   wherein the first liquid composition comprising urea which is provided in step (i) is part or all of the urea recyclate obtained in step (i)d. The dotted line on FIG. 5 illustrates a non-limiting number of example points of the process where the thiosulfate salt provided in step (ii) can be introduced.

Any solidification apparatus used in the solidification section of step (i)c will produce urea dust during normal operation which, if not treated, causes urea emissions in the surrounding atmosphere. Most solidification sections will by default collect the gas (typically air, also referred to as “solidifying gas”) which is used for cooling and solidifying the urea melt of step (i)b in order to submit the gas to treatment to at least reduce its ammonia content before it is vented to the atmosphere. Even in case of solidification apparatuses which do not explicitly rely on a forced solidifying gas stream, such as pan granulators or rotoformers, the ambient air has high urea dust and/or ammonia levels, which can be recovered from the solidification section using regular ventilation means. Most large-scale urea production facilities use a prilling tower or fluidized bed granulator which have dedicated air in- and outlets in order to actively force air flow. Since these plants already have a dedicated gas stream recovery from the solidification apparatus, the inventors have found that the method of the present invention is particularly preferred wherein step (i)c is performed in a prilling tower or fluidized bed granulator.

The present inventors have also found that in view of the valorisation of urea dust provided by the embodiments of the invention wherein step (i)d is performed and the first liquid composition comprising urea which is provided in step (i) is part or all of the urea recyclate obtained in step (i)d. there is no need to minimize dust formation from an environmental point of view (although some measures may still be desirable in view of fouling of process equipment). This has the additional advantage that the solid urea fertilizer produced in step (i)c can be produced substantially free of urea-formaldehyde, which allows the urea to be used not only as suitable for use as a fertilizer but also for other purposes, in particular as AdBlue® ingredient. Urea-formaldehyde is used as an antidusting agent to minimize dust formation during solidification but renders the urea unsuitable for certain other uses, such as AdBlue® ingredient. Hence, in preferred embodiments of the present invention the method described herein is provided wherein the urea melt of step (i)b is substantially free of urea-formaldehyde, preferably free of any antidusting agent.

The recovery in step (i)d of urea from the gas stream of step (i)c to obtain a urea recyclate may be performed using any solid-gas separation means suitable for separating urea dust from the gas stream. In accordance with preferred embodiments of the invention, the amount of urea recovered in step (i)d is within the range of 0.5-5% of the urea fed to the solidification section of step (i)c, preferably within the range of 2-5%. In case the method is operated as a continuous process, the amount of urea recovered in step (i)d within a predetermined timeframe is within the range of 0.5-5% of the urea fed to the solidification section of step (i)c in the same timeframe, preferably within the range of 2-5%. The urea recyclate is preferably a liquid aqueous composition comprising urea.

In a preferred embodiment of the invention, step (i)d is performed using cyclones and/or filters to obtain urea dust, and contacting said urea dust with an aqueous phase. In such embodiments, the urea recyclate is preferably obtained in the form of an aqueous composition comprising at least 20 wt. % (by total weight of the composition) urea and at least 30 wt. % (by total weight of the composition) water, preferably in the form of an aqueous composition comprising 20-45 wt. % (by total weight of the composition) urea and 55-80 wt. % (by total weight of the composition) water.

In an alternative, and more preferred embodiment of the invention, step (i)d is performed by means of a scrubber wherein the gas stream is contacted with an aqueous phase. In such embodiments the urea recyclate is preferably obtained in the form of an aqueous composition comprising at least 25 wt. % (by total weight of the composition) urea and at least 30 wt. % (by total weight of the composition) water, preferably in the form of an aqueous composition comprising at least 25-45 wt. % (by total weight of the composition) urea and 55-75 wt. % (by total weight of the composition) water. A scrubber is also interchangeably referred to herein as an “absorber”, which is equipment that permits rapid, intimate contact of gaseous process stream(s) and an aqueous medium, for example, a falling-film column, a packed column, a bubble column, a spray-tower, a gas-liquid agitated vessel, a plate column, a rotating disc contactor, a venturi tube, etc. The functioning of such absorbers is known to the skilled person, and in the case of vertical absorbers (e.g. columns, spray-towers) typically involves introducing one or more gas streams at the bottom part of the absorber, and introducing an aqueous phase at the top part of the absorber, such that the gas and the aqueous phase react in counter-current. The aqueous phase accumulates in the bottom part, where a level meter may monitor the liquid level and activate a pump to safeguard a maximum liquid level.

In some embodiments of the invention, the aqueous phase fed to the scrubber comprises an acid, such as oxalic acid, hydrochloric acid, sulphuric acid and/or nitric acid, preferably sulphuric acid and/or nitric acid, more preferably sulphuric acid, such that the urea recyclate obtained from the scrubber further comprises an ammonium compound selected from ammonium oxalate, ammonium chloride, ammonium sulphate, ammonium nitrate, and combinations thereof, preferably selected from ammonium sulphate, ammonium nitrate, and combinations thereof, preferably selected form ammonium sulphate. This ammonium compound will be comprised in the final urea-thiosulfate fertilizer next to at least urea and thiosulfate. In this way, for example a solid urea-thiosulfate fertilizer which is a combined urea-thiosulphate-sulphate/nitrate product can be produced.

In this way, for example a solid nitrogen-sulfur fertilizer which is a combined urea-thiosulphate-sulphate/nitrate/chloride/oxalate product can be produced. As is evident from the preferred embodiments outlined above, the use of hydrochloric acid and associated production of ammonium chloride is significantly less preferred than the use of oxalic acid, sulphuric or nitric acid and associated production of ammonium sulphate or nitrate, since chloride stress leads to corrosion issues in urea production plants. The use of oxalic acid or sulphuric acid and associated production of ammonium oxalate or sulphate is more preferred, with ammonium sulphate being most preferred.

However, it is highly preferred that the aqueous phase fed to the scrubber is substantially free of acid, in particular substantially free of oxalic acid, sulphuric acid, hydrochloric acid and nitric acid, such that an urea recyclate which is substantially free of ammonium compound selected from ammonium oxalate, ammonium chloride, ammonium sulphate, ammonium nitrate, and combinations thereof is obtained. This allows for maximum flexibility in adjusting the composition of the final urea-thiosulfate fertilizer obtained in step (v). For example, in some embodiments of the invention, no ammonium compound selected from ammonium oxalate, ammonium chloride, ammonium sulphate, ammonium nitrate, and combinations thereof is added to the urea recyclate stream such that the final nitrogen-sulfur fertilizer obtained in step (v) is substantially free of ammonium compound selected from ammonium oxalate, ammonium chloride, ammonium sulphate, ammonium nitrate, and combinations thereof. In other embodiments of the invention, the final nitrogen-sulfur fertilizer does comprise ammonium compound selected from ammonium oxalate, ammonium chloride, ammonium sulphate, ammonium nitrate, and combinations thereof, but by obtaining an urea recyclate which is substantially free of ammonium compound selected from ammonium oxalate, ammonium chloride, ammonium sulphate, ammonium nitrate, and combinations thereof from the scrubber, the concentration of ammonium compound can easily be controlled.

Accordingly, in some embodiments of the invention, step (i)d comprises contacting in a first scrubber the gas stream of step (i)c with an aqueous phase which is substantially free of acid, in particular substantially free of oxalic acid, hydrochloric acid, sulphuric acid and/or nitric acid such that an urea recyclate which is substantially free of ammonium compound selected from ammonium oxalate, ammonium chloride, ammonium sulphate and ammonium nitrate is obtained; recovering the off-gas from the first scrubber and contacting the off-gas from the first scrubber in a second scrubber with an aqueous phase comprising oxalic acid, hydrochloric acid, sulphuric acid and/or nitric acid, preferably sulphuric acid and/or nitric acid, more preferably sulphuric acid, such that an aqueous ammonium compound stream is obtained. The ammonium compound stream can then either be used for other purposes or, as in some preferred embodiments of the invention, at least part of the ammonium compound stream is combined with the urea composition provided in step (i)before the solidification of step (v) and before, during and/or after combining the urea composition provided in step (i) with the thiosulfate composition i.e. thiosulfate salt provided in step (ii), such that the final urea-thiosulfate fertilizer obtained in step (v) comprises an ammonium compound selected from ammonium oxalate, ammonium chloride, ammonium sulphate, ammonium nitrate, and combinations thereof, preferably ammonium sulphate next to at least urea and thiosulfate.

In particularly preferred embodiments of the invention, step (i)d is performed and the first liquid composition comprising urea which is provided in step (i) is part or all of the urea recyclate obtained in step (i)d; step (i)d comprises separating urea dust from the gas stream by means of a scrubber as described herein earlier; concentration step (iv) is performed by evaporation as described herein earlier; and at least part of the condensate from the evaporator is recirculated to the scrubber of step (i)d to form at least part of the aqueous phase fed to the scrubber. In alternative embodiments of the invention, step (i)d is performed and the first liquid composition comprising urea which is provided in step (i) is part or all of the urea recyclate obtained in step (i)d; step (i)d comprises recovering urea from the gas stream by means of cyclones and/or filters, thereby obtaining urea dust, and contacting said urea dust with an aqueous phase as described herein earlier; concentration step (iv) is performed by evaporation as described herein earlier; and at least part of the condensate from the evaporator is recirculated to step (i)d to form at least part of the aqueous phase used to dissolve the urea dust.

It is particularly preferred that the embodiments of the invention described herein elsewhere specifying recycling of non-converted reagents from a recovery section in step (i)a and recycling of evaporator condensate from the evaporator of step (i)b to step (i)a are combined with the recycling of condensate of the evaporator of step (iv) to the urea recovery of step (i)d as described in the previous paragraphs. In this way an optimized and efficient process can be obtained.

In some embodiments, step (v) of the method of the present invention further comprises submitting the solid urea-thiosulfate fertilizer to a drying step. The present inventors have found that this may be useful to eliminate trace moisture from the solid urea-thiosulfate fertilizer.

The method of the present invention may be operated in batch, semi-continuous or continuous mode, but is preferably operated in continuous mode.

In all embodiments of the method described herein, it is preferred that the solid urea-thiosulfate fertilizer comprises urea and thiosulfate in an amount such that the ratio (w/w) of “N from urea” to “S from thiosulfate” is at most about 8:1, preferably at most about 7.5:1, more preferably at most about 7:1, and at least about 1.1:1, preferably at least about 1.5:1, more preferably at least about 2:1, wherein N refers to the total amount of nitrogen (N) from urea in the solid urea-thiosulfate fertilizer, and S refers to the total amount of sulfur (S) from thiosulfate in the solid urea-thiosulfate fertilizer.

### EXAMPLES

### Example 1: Melting Point Depression of Urea—Ammonium Thiosulfate Blends

An ammonium thiosulfate solution (Thio-Sul® from Tessenderlo Kerley) was freeze dried to obtain solid ammonium thiosulfate, which was examined by Differential Scanning calorimetry. The melting point of solid ammonium thiosulfate was determined to be about 134° C.

The melting point for different urea-ammonium thiosulfate blends was determined by mixing urea, ammonium thiosulfate (freeze dried from Thio-Sul®) and optionally water at different ratios to a total combined amount of 70 g, heating the mixture and determining the melting point by visual observation. The results are shown in FIG. 6, wherein the x-axis shows the urea wt. % on total solids basis. As can be seen in FIG. 6, the urea-ammonium thiosulfate blends exhibit a significant melting point depression at 70:30 and at 80:20 urea:ammonium thiosulfate (w/w) ratio's. The presence of water in an amount of 3-10 wt. % in the urea-ammonium thiosulfate blends decreased the melting point temperature.

As a comparison a urea-ammonium thiosulfate blend having a 99:1 urea:ammonium thiosulfate (w/w) ratio was similarly prepared. This urea-ammonium thiosulfate blend was solid at 128° C. and the melting point depression was insufficient at this composition.

Similar tests were performed using calcium thiosulfate and a melting point depression was also observed.

A comparison is visualized with melting temperatures of urea/ammonium sulfate blends obtained from literature with the experimentally determined urea/ammonium thiosulfate blend melting temperatures.

The results are shown in FIG. 7. Is can clearly be seen that the melting temperature of urea/ammonium thiosulfate blends decreases further with increasing ammonium thiosulfate content in comparison to the melting temperature of urea/ammonium sulfate which increases above 10 solid wt % ammonium sulfate.

### Example 2: Stability of Urea—Ammonium Thiosulfate Blends

An ammonium thiosulfate solution (Thio-Sul® from Tessenderlo Kerley) was freeze dried to obtain solid ammonium thiosulfate, which was blended with grinded urea powder into a homogeneous mixture. A master batch of the urea-ammonium thiosulfate blend having a 80:20 urea:ammonium thiosulfate (w/w) ratio was prepared and divided into different glass jars, containing 20 g powder blend each, which were all placed into an oil batch at a stable temperature of either 140° C. or 110° C. One glass jar was removed after a certain residence time being: 1 min, 2 min, 3 min, 5 min, 10 min and 15 min. The material was left to solidify at room temperature. Decomposition was follow by determining the ammonium thiosulfate content by means of titration for every sample.

It can be seen from the results shown in FIG. 8 that ammonium thiosulfate decomposition is significantly lower at 110° C. than at 140° C. Hence, thanks to the melting point depression found with certain urea—ammonium thiosulfate blends described herein, enabling lower processing temperatures than for regular urea melts, the processing and provision of urea—ammonium thiosulfate blends is greatly facilitated.

Another urea—ammonium thiosulfate blend was prepared having a 80:20 urea:ammonium thiosulfate (w/w) ratio as described above with the exception that the material was solidified through fluid bed granulation thereby obtaining granules. This urea—ammonium thiosulfate blend was divided into different glass jars, containing 20 g granular blend each, which were all placed into an oil batch at a stable temperature of either 110° C., 120° C., 125° C., 130° C. or 140° C. One glass jar was removed after a certain residence time being: 1 min, 2 min, 3 min, 5 min, 10 min and 15 min. The material was left to solidify at room temperature. Decomposition was follow by determining the ammonium thiosulfate content by means of titration for every sample.

It can be seen from the results shown in FIG. 9 that ammonium thiosulfate decomposition is significantly lower at a temperature of <130° C. than at 140° C. Hence, despite this blend being in a different form the melting point depression was also found with certain urea—ammonium thiosulfate blends described herein, to enable lower processing temperatures than for regular urea melts, which greatly facilitates the processing and provision of urea—ammonium thiosulfate blends.

An urea-calcium thiosulfate blend having a 80:20 urea:calcium thiosulfate (w/w) ratio was similarly prepared to the urea—ammonium thiosulfate blend by freeze drying of CaTs to obtain a dry powder product to prepare a physical blend with grinded urea. The urea-calcium thiosulfate blend had 20 g of the powder blend added to glass jars, which was placed in an oil bath at 125° C. One glass jar was removed after a certain residence time being: 5 min, 10 min and 15 min. The material was left to solidify at room temperature. Decomposition was follow by determining the calcium thiosulfate content by means of titration for every sample. It can be seen from the results shown in FIG. 10 that calcium thiosulfate decomposition is stable at a temperature of 125° C. within the standard error margin of +−0.5 CaTs % of the titration method.

An urea-potassium thiosulfate blend having a 80:20 urea:potassium thiosulfate (w/w) ratio was similarly prepared to the urea—ammonium thiosulfate blend by freeze drying of potassium thiosulfate to obtain a dry powder product to prepare a physical blend with grinded urea. The urea-potassium thiosulfate blend had 20 g of the powder blend added to glass jars, which was placed in an oil bath at 125° C. One glass jar was removed after a certain residence time being: 5 min, 10 min and 15 min. The material was left to solidify at room temperature. Decomposition was follow by determining the potassium thiosulfate content by means of titration for every sample. It can be seen from the results shown in FIG. 11 that potassium thiosulfate decomposition is also stable at a temperature of 125° C.

### Example 3: Evaporation of Urea—Ammonium Thiosulfate Blends

Wiped-film evaporation tests were carried out on liquid urea—ammonium thiosulfate blends having a 80:20 urea:ammonium thiosulfate (w/w) ratio prepared by combining 70 wt % urea liquid and ammonium thiosulfate solution (Thio-Sul® from Tessenderlo Kerley, 55 wt % ATS). The wiped-film evaporation tests involved adjusting the parameters of heating oil temperature, residence/time throughput and vacuum pressure. Table 1 below shows a number of liquid UATS blends. However, only tests 5 and 6 in Table 1 were suitable for melt fluid bed granulation to obtain a fertilizer of good quality, because of the blends having low water content and minimal to no loss of ammonium thiosulfate through decomposition during the evaporation step.

TABLE 1

| | | Residence | | | Decomposition- | Relative |
|---|---|---|---|---|---|---|
| | Oil | time/ | Vacuum | Remaining | insoluble | ATS % |
| Test | temperature | throughput | pressure | water % | content | loss |
| 1 | 130° C. | 8 kg/h | 750 mbar | >4 wt % | | |
| 2 | 130° C. | 8 kg/h | 400 mbar | >4 wt % | | |
| 3 | 130° C. | 8 kg/h | 50 mbar | <4 wt % | Non-acceptable | >10% |
| 4 | 140° C. | 12 kg/h | 50 mbar | <4 wt % | Non-acceptable | >10% |
| 5 | 120° C. | 8 kg/h | 50 mbar | <4 wt % | Acceptable | <10% |
| 6 | 120° C. | 20 kg/h | 50 mbar | <4 wt % | Acceptable | <10% |
| 7 | 130° C. | 20 kg/h | 50 mbar | <4 wt % | Non-acceptable | >10% |

1. A method for the production of a solid urea-thiosulfate fertilizer, said method comprising the following steps:
(i) providing a first liquid composition comprising urea;
(ii) providing a second composition comprising a thiosulfate salt;
(iii) combining the composition provided in step (i) with the composition provided in step (ii) to obtain a liquid urea-thiosulfate blend comprising 70-90 wt. % (by dry weight of the urea-thiosulfate blend) urea and 10-30 wt. % (by dry weight of the urea-thiosulfate blend) thiosulfate salt;
(iv) optionally submitting the liquid urea-thiosulfate blend of step (iii) to a concentration step to obtain a concentrated liquid urea-thiosulfate blend; and
(v) feeding the liquid urea-thiosulfate blend of step (iii) and/or the concentrated liquid urea-thiosulfate blend of step (iv) as a liquid at a temperature of less than 128° C. to a solidification section wherein the liquid is converted to a particulate solid, thereby obtaining the solid urea-thiosulfate fertilizer.

2. The method claim 1, wherein the first liquid composition provided in step (i) is an urea melt comprising at least 95 wt. % (by total weight of the composition) urea, preferably comprising at least 96 wt. % (by total weight of the composition) urea.

3. The method according to claim 1, wherein the first liquid composition provided in step (i) is an aqueous composition comprising 25-45 wt. % (by total weight of the first liquid composition) urea and 55-75 wt. % (by total weight of the first liquid composition) water.

4. The method according to claim 1, wherein the first liquid composition provided in step (i) is an aqueous composition comprising 65-95 wt. % (by total weight of the first liquid composition) urea and at least 5 wt. % (by total weight of the first liquid composition) water.

5. The method according to claim 1, wherein step (iv) is applied and wherein the concentrated liquid urea-thiosulfate blend obtained in step (iv) has a water content of less than 5 wt. % (by total weight of the concentrated blend), preferably less than 4 wt. % (by total weight of the concentrated blend).

6. The method according to claim 5, wherein step (iv) comprises concentrating the liquid urea-thiosulfate blend in an evaporator.

7. The method according to claim 6, wherein step (iv) is performed at a temperature of less than 128° C., preferably a temperature within the range of 100-125° C.

8. The method according claim 1, wherein the method is integrated with a urea production facility, step (i) comprising the following steps:
(i)a synthesizing urea from ammonia and carbon dioxide thereby obtaining a liquid aqueous composition comprising urea;
(i)b optionally concentrating the aqueous composition obtained in step (i) to obtain a liquid urea melt comprising more than 95 wt. % (by total weight of the melt) urea
(i)c optionally submitting the urea melt of step (i)b to a solidification step in a solidification section wherein the melt is converted to a particulate solid, thereby obtaining a solid urea fertilizer, and optionally recovering from the solidification section a gas stream comprising urea, such as urea dust; and
(i)d optionally separating urea from the gas stream recovered from the solidification section, thereby obtaining an urea recyclate which is preferably a liquid aqueous composition comprising urea, more preferably the urea recyclate is an aqueous composition comprising 30-50 wt. % urea (by total weight of the composition) and at least 40 wt. % water;
wherein the first liquid composition comprising urea which is provided in step (i) is the liquid aqueous composition obtained in step (i)a; the liquid urea melt obtained in step (i)b and/or the urea recyclate obtained in step (i)d.

9. The method according to claim 8, wherein the urea production facility is first operated in a first operational mode wherein
part or all of the liquid aqueous composition obtained in step (i)a is provided in step (i) for the production of a urea-thiosulfate based fertilizer, and steps (i)b, (i)c and (i)d are not performed; or
part or all of the liquid urea melt obtained in step (i)b is provided in step (i) for the production of a urea-thiosulfate based fertilizer, and steps (i)c and (i)d are not performed;
and wherein after completion of the production of urea-thiosulfate based fertilizer according to steps (i)-(v), the urea production facility is subsequently operated in a second operational mode wherein all of the liquid aqueous composition obtained in step (i)a is used for the production of a particulate solid which comprises more than 95 wt. % (by dry weight of the particulate solid) urea, preferably more than 98 wt. % (by dry weight of the particulate solid) urea according to steps (i)b-(i)c.

10. The method according to claim 9, wherein the concentration step (iv) is performed in an evaporator and the second operational mode employs an evaporator in step (i)b which is distinct from the evaporator which is used in step (iv) of the first operational mode.

11. The method according to claim 8, which is for the coproduction of urea fertilizer and urea-thiosulfate fertilizer in an urea production facility, wherein steps (i)b, (i)c, and optionally (i)d are performed and wherein the first liquid composition comprising urea which is provided in step (i) is part of the liquid aqueous composition obtained in step (i)a; part of the liquid urea melt obtained in step (i)b and/or part or all of the urea recyclate obtained in step (i)d.

12. The method according to claim 11, which is for the simultaneous coproduction of urea fertilizer and urea-thiosulfate fertilizer in an urea production facility wherein steps (i)b and (i)c are performed simultaneously with the production of urea-thiosulfate based fertilizer according to steps (i)-(v), wherein
step (iv) comprises concentrating the liquid urea-thiosulfate blend in a first evaporator;
step (i)b comprises concentrating the liquid composition of step (i)b in a second evaporator,
wherein the second evaporator employed in step (i)b is a distinct apparatus from the first evaporator employed in step (iv); and
preferably the solidification section of step (v) comprises a first solidification apparatus and step (i)c is performed employing a second solidification apparatus distinct from the first solidification apparatus of step (v).

13. The method according to claim 12, wherein the second composition provided in step (ii) is provided in the form of an aqueous solution of thiosulfate salt, wherein step (iv) is performed, and wherein step (iv) comprises concentrating the liquid urea-thiosulfate blend of step (iii) to obtain a concentrated liquid urea-thiosulfate blend having a water content of less than 5 wt. % (by total weight of the concentrated liquid urea-thiosulfate blend), preferably less than 4 wt. % (by total weight of the concentrated liquid urea-thiosulfate blend).

14. The method according to claim 1, wherein the solidification section of step (v) comprises a solidification apparatus selected from selected from a prill tower, a rotoformer, a drum granulator and a fluidized bed granulator.

15. The method according to claim 1, wherein the thiosulfate salt is ammonium thiosulfate.
